(19) United States
(12) Patent Application Publication
(10) Pub. No.: US 2021/0059899 A1
(43) Pub. Date: Mar. 4, 2021

(54) ANTEROPOSTERIOR THORACIC RESTRICTION DEVICE

(71) Applicants: INSERM (INSTITUT NATIONAL DE LA SANTÉ ET DE LA RECHERCHE MÉDICALE), PARIS (FR); ASSISTANCE PUBLIQUE - HÔPITAUX DE PARIS, PARIS (FR); UNIVERSITE PARIS-EST CRETEIL VAL DE MARNE, CRETEIL (FR)

(72) Inventors: ARMAND MEKONTSO DESSAP, PARIS (FR); GUILLAUME CARTEAUX, MONTREUIL (FR)

(21) Appl. No.: 16/644,530

(22) PCT Filed: Sep. 5, 2018
(86) PCT No.: PCT/FR2018/052163

§ 371 Date: Sep. 11, 2020

(30) Foreign Application Priority Data
Sep. 5, 2017 (FR) 1758169

(51) Int. Cl. A61H 31/00; A61F 5/34; A61F 5/32; A61M 16/06; A61M 16/04; A61H 9/00

(52) U.S. Cl. CPC A61H 31/00; A61F 5/34; A61F 5/32; A61M 2016/0027; A61M 16/0666; A61M 16/04; A61H 9/0078; A61M 16/06

(57) ABSTRACT

The invention relates to an anteroposterior thoracic restriction device comprising holding means intended to surround a patient's chest, a compressible fluid bag, intended to be held against the patient's sternum by said holding means, and reversible bilateral tightening means, arranged on either side of the fluid bag and capable of reversibly tightening the holding means around the patient's chest.

The invention relates to an anteroposterior thoracic restriction device for surrounding the chest of a patient to minimize positive-pressure ventilation in the anterior portion of the lungs of said patient and to promote redistribution of positive-pressure ventilation to the posterior portion. The invention has applications in the medical field, particularly in the field of ventilatory support for patients with respiratory pathology associated with inhomogeneous pulmonary lesions. The invention is particularly suitable for the treatment or prevention of acute or chronic respiratory insufficiency.

Ventilatory support by positive intrathoracic pressure is classically used to compensate for a patient's respiratory insufficiency. A respiratory interface, either invasive such as an intubation tube, or noninvasive such as a mouth, nose or face mask, is then applied to the patient, and is connected to a pressure generator (commonly called a ventilator) to artificially infuse air into the lungs. This global delivery of ventilatory support is however unsuited to the distribution of the pulmonary lesion. Indeed, the distribution of lung lesions is generally inhomogeneous: there are typically condensed nonventilated areas with generally posterior distribution, and healthy anterior aerated areas. This distribution, particularly observed during acute respiratory distress syndrome (ARDS), is explained in particular by the weight of the heart, the compression of the overlying lung due to gravity, and the natural inhomogeneity of the compliance of the thoracic wall, which is more important in the anterior part than in the posterior part, especially when the patient is in dorsal decubitus position. Thus, when ventilatory support is delivered globally, ventilation is distributed preferentially in areas that are already ventilated. This exposes to a lack of ventilation of condensed areas (then exposed to so-called opening-closing lesions), and an excess of ventilation of aerated areas (then exposed to so-called overdistension lesions). Opening-closing and overdistension lesions aggravate preexisting lung damage and lead to excess mortality in patients.

In patients with ARDS, the ventral decubitus position (lying on the stomach) tends to rehomogenize ventilation through various physiological effects, in particular reduced compliance of the anterior chest wall. It has been shown that the patient's ventral decubitus prone position for at least 16 hours every 24 hours decreases mortality during ARDS.

However, the ventral decubitus position is very little practiced in patients under artificial ventilation with ARDS, due to the cumbersome nature of the technique, the frequency of associated organ failures which limit its feasibility, and the side effects observed (pressure sores on the face, in particular).

There is today no device to limit overdistension of properly aerated areas of the lungs and to promote redistribution of ventilation to condensed areas in a patient requiring artificial ventilatory support.

The objective of the invention is to at least partially solve the problem associated with the excessive distribution of ventilation in the aerated areas of the lungs to the detriment of the condensed areas in a patient in dorsal decubitus position in need of artificial ventilatory support. For this purpose, the invention proposes a device to be applied against the thoracic cage of the patient in dorsal decubitus position, making it possible to maximize ventilatory support in the posterior areas of the lungs and to minimize it in the anterior areas. More precisely, the device according to the invention comprises a compression interface such as a fluid bag intended to be held in close contact opposite the patient's sternum, so as to apply a positive extrathoracic pressure located on the anterior part of the thoracic wall, opposite the pulmonary areas which are generally correctly aerated and therefore exposed to overdistension. This device specifically reduces the compliance of the anterior chest wall, thus homogenizing the transpulmonary pressure and thus increasing the load on the posterior part of the lungs. Bilateral tightening means are used to apply measured pressure to the fluid bag opposite the sternum. According to another aspect, the device according to the invention comprises at least one tightening means for applying a pressure measured in the fluid bag located opposite the sternum.

The invention therefore has as its object an anteroposterior thoracic restriction device comprising at least one holding means intended to surround the thoracic cage of a patient, at least one compressible fluid bag, intended to be held against the sternum of the patient by at least one holding means, and reversible bilateral tightening means, arranged on either side of the fluid bag and capable of reversibly tightening at least one holding means around the thoracic cage of the patient.

In the context of the invention, the term “patient” means a mammal, and preferentially a human, including an adult, child or infant. The term “patient” can also refer to a nonhuman animal, particularly a nonhuman primate.

According to the invention, the one or more holding means hold the compressible fluid bag against the patient's sternum. In addition, said holding means must have a low compliance so as to apply a homogeneous pressure on the fluid bag.

The bilateral tightening means allow the one or more holding means to be tightened evenly and in a controlled manner around the patient's chest. Thus, the pressure exerted is homogeneous on both sides of the chest. Of course, if necessary, it is possible to tighten the bilateral tightening means in different ways, in order to influence the pressure on both sides of the chest.

In one embodiment the holding means comprise a strap, preferentially a semi-rigid strap.

Alternatively or additionally, the holding means comprise a rigid or semi-rigid anterior plate intended to be applied against the anterior part of the patient's chest, and optionally a rigid or semi-rigid posterior plate intended to be applied against the posterior part of the patient's chest. The anterior and/or posterior plate can be multi-perforated in order to adapt the position of the bilateral tightening means to the diameter of the patient's chest.

The fluid bag is then compressed between the anterior plate and the patient's chest wall.

In an embodiment, the anterior plate is in one piece. In another embodiment, the anterior plate consists of several elements superimposed one on top of the other.

It is then possible to adjust the number of elements to play with the thickness of the anterior plate.

In an embodiment, the holding means further comprise lateral holding means or side tabs for connecting the anterior plate to the tightening means. These lateral holding means can be flexible, rigid or semi-rigid and extend advantageously on either side of the anterior plate. Preferably, the lateral holding means are arranged in the extension of the anterior plate and are possibly raised in relation to the anterior plate. In an embodiment, reinforcements are provided to stiffen the side tab system. These reinforcements may be L- or T-shaped in cross-section. Preferably, the longitudinal edges of the side tabs may be profiled so that their cross-sections are L- or T-shaped in order to increase the stiffness of the side tabs in the longitudinal direction.

Advantageously, the side tabs are semi-rigid or rigid, in order to optimally transmit the pressure exerted by the bilateral tightening means. Preferentially, the side tabs are also raised above the anterior plate so that they do not come into contact with the patient's skin when the device is inserted, regardless of the patient's morphotype and sex.

In one embodiment, the dimensions of the anterior plate are substantially equal to those of the fluid bag, so that said plate covers only said bag. This embodiment maintains access to the patient for monitoring (in particular when patches connected to a monitoring screen are adhered on the anterior face of the patient's thorax), for clinical examination and in particular auscultation. It also limits the risk of contact of the anterior plate with the patient's skin and thus the risk of skin intolerance and pressure sores.

In another embodiment, the dimensions of the anterior plate are strictly larger than those of the compressible fluid bag so that the plate extends on either side of the bag so as to at least partially cover the patient's chest.

Advantageously, the tightening means are progressive tightening means, in order to control and adapt more precisely the pressure applied to the anteroposterior part of the chest. In an embodiment, the tightening means are anterolateral. Preferentially, the device also comprises means for automatic release, so that the patient's chest can be released quickly and effortlessly. According to another embodiment and when using a tightening means, the device may have only one automatic release device.

Advantageously, the one or more tightening means are able to apply a pressure of between 20 and 150 cm of water (cmH₂O), preferably 60 cmH₂O, ±20 in the fluid bag, when the latter is held between the holding means and the chest of a patient.

In an embodiment, the anteroposterior thoracic restriction device further comprises a pressure sensor for measuring the pressure in the fluid bag. It is then possible to precisely control the extrathoracic pressure applied.

In an embodiment, the fluid bag contains a liquid, preferentially water. Of course, it is possible to use another fluid, and in particular a gas.

Advantageously, the amount and/or volume of fluid contained in the bag is constant. “Constant” means that the amount and/or volume of fluid in the bag at constant temperature and pressure is fixed, i.e. does not vary, preferentially at room temperature (i.e., 20 to 30° C.) and atmospheric pressure. The bag contains a defined and constant amount of fluid, at least for the duration of the use of the thoracic restriction device. Thus, the patient's thoracic compression results from the use of the tightening means and not from variations in the amount and/or volume of fluid contained in the compressible bag. Preferentially, the constant fluid volume contained in the bag is approximately 1 liter or 10⁶ mm³.

In an embodiment, the size of the fluid bag is approximately equal to the dimensions of the patient's sternum. Alternatively, the dimensions of the bag are such that the bag extends slightly beyond the dimensions of the patient's sternum, particularly on part of the ribs on either side of the sternum. In general, the dimensions of the compressible fluid bag are such that it cannot extend over the lateral and dorsal portions of the chest.

In an embodiment, the holding means comprise a central housing for receiving the fluid bag. For example, the holding means comprises a strap intended to surround the chest and a housing is provided in the inner front part of the strap, intended to be applied against the chest wall. In another example, the holding means comprise an anterior plate for compressing the anterior chest and a housing is provided on the inside of the plate for application against the chest wall.

The invention also relates to an artificial ventilation system comprising a ventilator connected to a nasal and/or buccal and/or tracheal interface, for bringing air into the lungs of a patient, said system further comprising an anteroposterior thoracic restriction device according to the invention.

Another object of the invention is an artificial ventilation kit comprising such an artificial ventilation system, and means for depressing at least part of the patient's chest.

The invention will be better understood upon reading the following description and examining the accompanying figures. These are presented by way of non-limiting illustration of the invention. The figures represent:

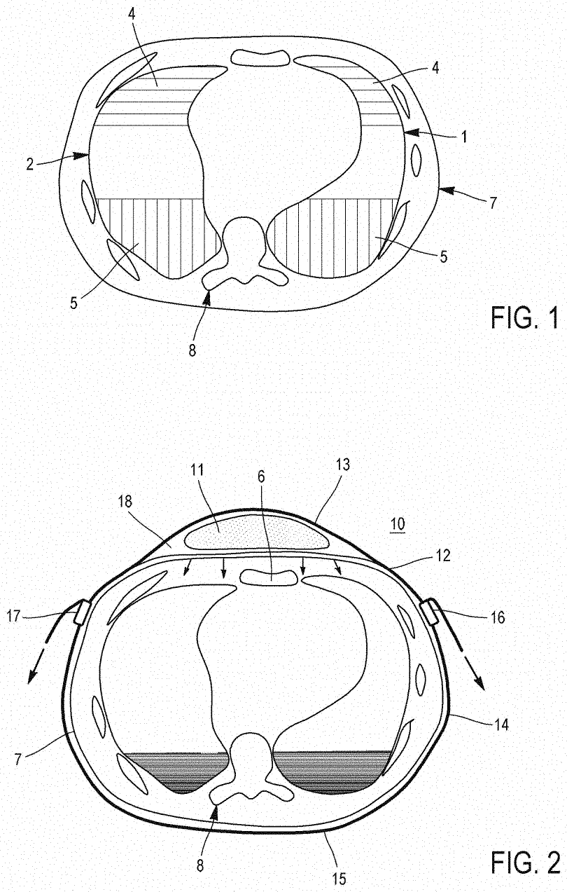

FIG. 1: a schematic cross-sectional representation of the chest of a patient with inhomogeneous lesions, with condensation of the posterior part of the right and left lungs;

FIG. 2: a schematic cross-sectional representation of the thoracic cage of a patient on which an anteroposterior thoracic restriction device is held according to a first example embodiment of the invention;

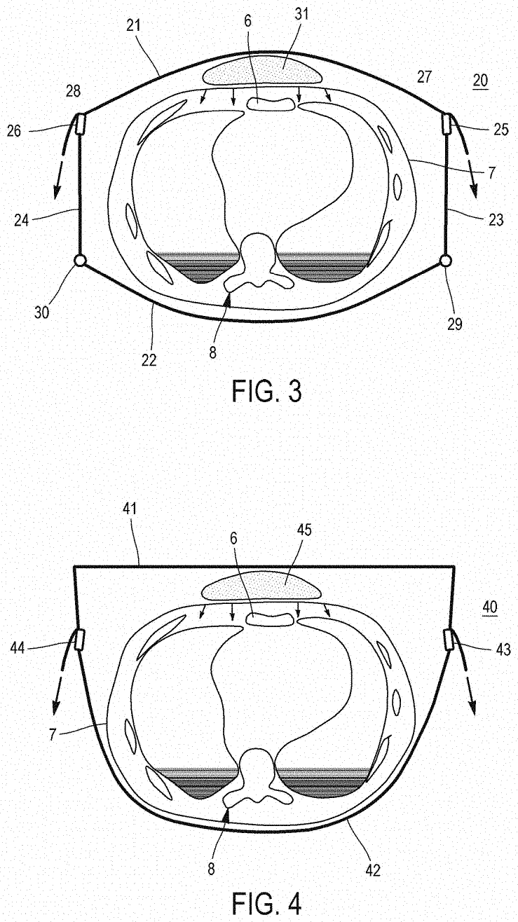

FIG. 3: a schematic cross-sectional representation of the thoracic cage of a patient on which an anteroposterior thoracic restriction device is held according to a second example embodiment of the invention;

FIG. 4: a schematic cross-sectional representation of the thoracic cage of a patient on which an anteroposterior thoracic restriction device is held according to a third example embodiment of the invention;

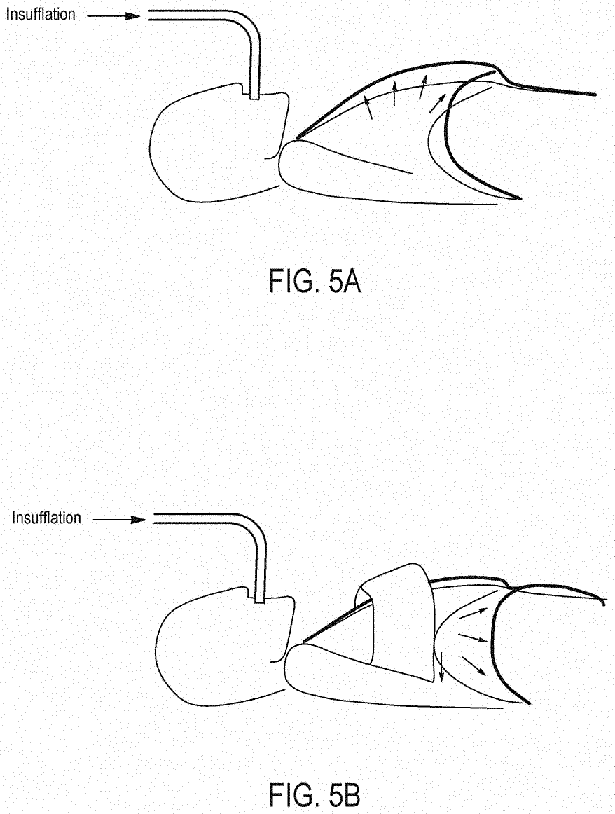

FIG. 5: a schematic representation of the essentially anterior distribution of positive-pressure artificial ventilation in a patient without the device according to the invention (A) and the posterior and inferior redistribution of positive-pressure artificial ventilation in a patient with the device according to the invention (B).

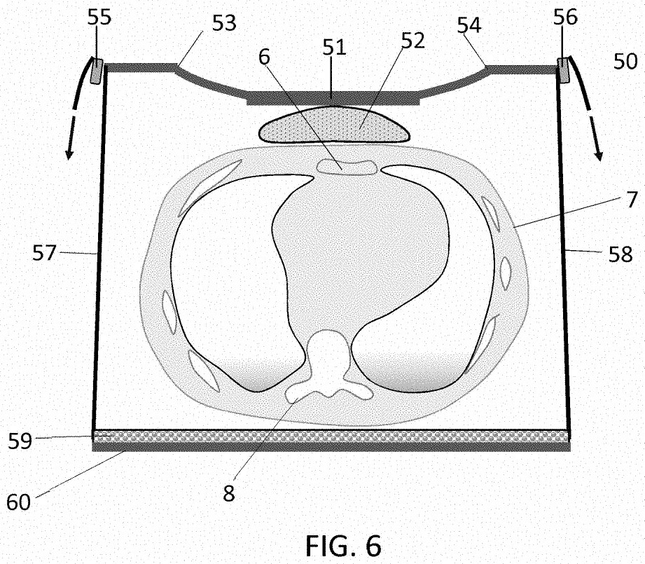

FIG. 6: a schematic cross-sectional representation of the thoracic cage of a patient on which an anteroposterior thoracic restriction device is held according to a fourth example embodiment of the invention;

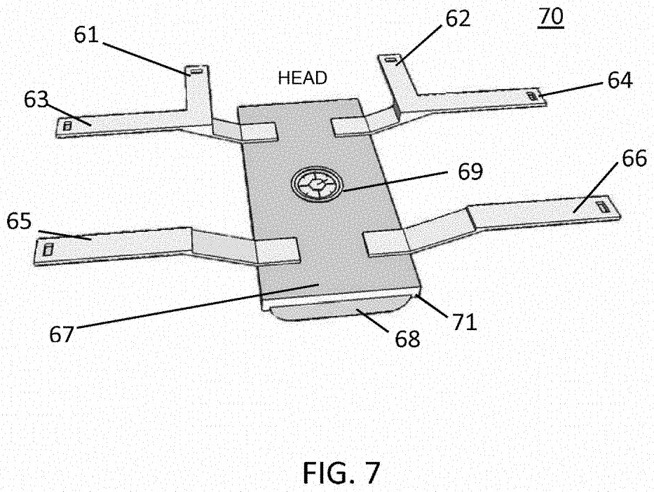

FIG. 7: a schematic representation of a thoracic restriction device according to an example embodiment of the invention.

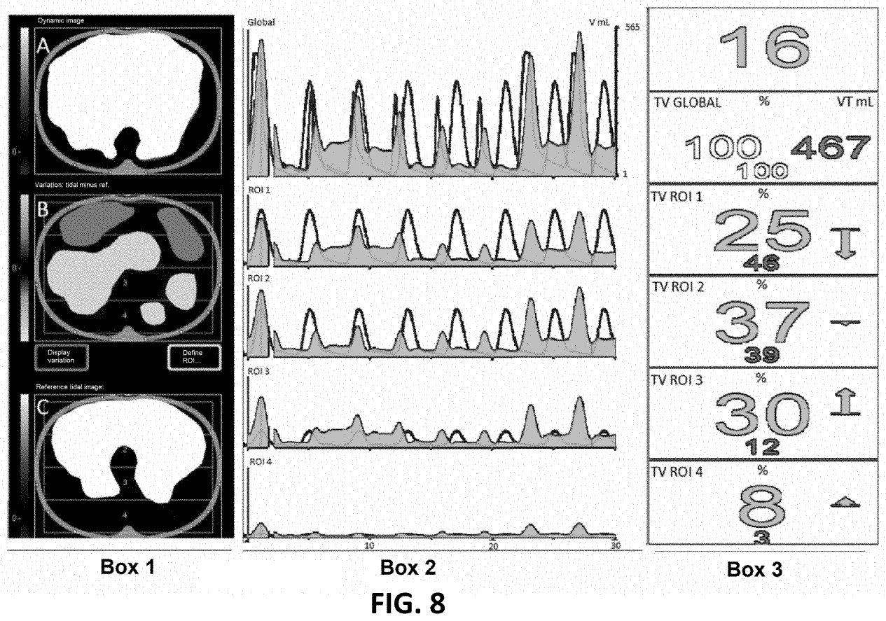

FIG. 8: a screenshot of a thoracic electrical impedance tomography (EIT) device of a mechanically ventilated cadaver (Thiel model) including the device according to FIG. 4. Box 1 describes dynamic EIT records of the Thiel model. Box 2 describes the overall impedance changes and impedance changes by region of interest, from the most anterior region (ROI 1) to the most posterior region (ROI 4) as a function of time. Box 3 describes the percentage of the total ventilation that reaches a region of interest.

As described above, some patients with respiratory failure have inhomogeneous lung lesions 1, 3. FIG. 1 shows a cross-section of a human patient's chest with the classic inhomogeneous distribution of lung lesions, lying supine (in the dorsal decubitus position), vertebrae 8 down in the figure. The distribution of global ventilation under positive-pressure leads to overdistension of the anterior areas 4 of the lungs 1, 2 and a lack of ventilation or poor ventilation of the posterior condensed areas 5 of the lungs, exposed to opening-closing lesions.

The device according to the invention makes it possible to alleviate this problem by limiting ventilation in the aerated anterior part of the lungs and by promoting the mobilization of the posterior part of the lungs. More precisely, the device according to the invention makes it possible to apply a positive extrathoracic pressure to the anterior part of the thoracic cage where said device is applied, resulting in a regional decrease in transpulmonary pressure. This regional decrease in the highest point in the lung pressure actually tends to homogenize the lung pressure throughout the lungs. When positive-pressure artificial ventilation is administered simultaneously to the patient, the anterior aerated lung areas are protected from overdistension injury by the regional decrease in transpulmonary pressure, and ventilation tends to redistribute to the posterior regions due to the homogenization of the transpulmonary pressure, all the more so as the ventilation mode is volume-controlled (i.e. insufflation of a preset tidal volume at a prescribed frequency until the set volume is reached, without the patient's participation and without taking into account his or her respiratory activity). FIGS. 2 to 4 and 6 schematically represent cross sections of a thoracic cage of a human patient, in dorsal decubitus position, provided with different embodiments of the anteroposterior thoracic restriction device according to the invention, suitable for limiting the overdistension of the anterior areas and for promoting the posterior mobilization of the lungs during positive-pressure artificial ventilation.

In FIG. 2, the anteroposterior thoracic restriction device 10 comprises a fluid bag 11 held against the patient's sternum 6 by means of a strap 12, forming holding means, which surrounds the patient's chest 7. The strap 12 is made of rigid or semi-rigid material, so that the low compliance of these holding means allows sufficient pressure to be applied by the bag 11 against the patient's sternum 6. In an embodiment, the strap 12 is made of a biocompatible material, such as polyurethane. “Biocompatible material” means a material suitable for use in or on biological tissues, without degrading the biological tissues involved or triggering allergic reactions during or after contact. In the context of the invention, the biocompatible material used must in particular take into account the properties of the patient's skin. In another embodiment, the strap 12 is made of leather. It is possible to use a strap of varying thickness, in particular a strap with a greater thickness in the area of contact 13 with the fluid bag 11 than in the anterolateral 14 and posterior 15 areas.

Independent bilateral tightening means 16, 17, arranged at the anterolateral levels of the chest 7, allow the strap 12 to be tightened around the patient's chest 7 to increase the positive extrathoracic pressure on the sternum 6 through the fluid bag 11.

Advantageously, the fluid bag 11 is held in position on the strap 12 so that it is not moved during use. For example, as shown in FIG. 2, the strap 12 has a housing 18 into which the fluid bag 11 can be inserted and held.

In the example embodiment shown in FIG. 3, the anteroposterior thoracic restriction device 20 comprises two semi-rigid plastrons made of preferentially biocompatible material, anterior 21 and posterior 22 respectively, forming the holding means. The anterior plastron 21 is applied against the anterior portion of the patient's chest, while the posterior plastron 22 is applied against the posterior portion of the patient's chest. Tightening means are used to hold the holding means in position on the patient's chest. More specifically, the tightening means comprise two tightening straps 23, 24, each associated with independent tightening systems 25, 26. The tightening straps 23, 24 each connect a lateral end 27, 28, of a first plastron 21 to a lateral end 29, 30, of the second plastron 22.

A fluid bag 31 is compressed between the anterior plastron 21 and the sternum 6 of the patient's chest 7. Of course, it is possible to use a thoracic restriction device that comprises only the anterior plastron. In this case, the tightening means comprises a tightening strap that completely surrounds the posterior and lateral parts of the patient's chest.

In the example embodiment shown in FIG. 4, the anteroposterior thoracic restriction device 40 comprises a rigid anterior plate 41 made of preferentially biocompatible material. The tightening means include a tightening strap 42 completely surrounding the anterolateral and posterior parts of the patient's chest 7. The tightening means also comprise independent bilateral tightening systems 43, 44. A fluid bag 45 is compressed between the anterior plate 41 and the sternum 6 of the patient's chest 7.

In an embodiment, the holding means include shoulder straps, preferentially adjustable in height. The shoulder straps are, for example, fixed at the top of the strap 12, the plastron(s) 21, 22 or the anterior plate 41. The shoulder straps prevent the support means and fluid bag from sliding down the patient's body, thus helping to keep the fluid bag in position on the sternum. The shoulder straps can consist of, or include, hook-and-loop textile strips (such as Velcro® strips), which can be easily adjusted in length and repositioned.

In the example embodiment shown in FIG. 6, the anteroposterior thoracic restriction device 50 comprises a rigid or semi-rigid anterior plate 51 made of a preferentially biocompatible material, for example a biocompatible plastic. A fluid bag 52 is compressed between the anterior plate 51 and the sternum 6 of the patient's chest 7. The anterior plate 51 is connected to the tightening means by lateral holding means 53, 54.

The lateral holding means 53, 54 can be made of semi-rigid or rigid material, such as plastic or metal such as aluminum. If they are made of semi-rigid material, it is possible to provide reinforcements to further stiffen them.

The tightening means comprise tightening straps 57, 58 connecting the lateral holding means to a posterior anchoring point without coming into contact with the patient's chest 7. The tightening means also comprise independent bilateral tightening systems 55, 56.

In the example shown in FIG. 6, the tightening means are connected to a rigid or semi-rigid posterior plate 60 held against the dorsal part of the patient's chest. This posterior plate 60 is advantageously covered on its side intended to be in contact with the patient's skin with a material designed to optimize skin tolerance and limit the risk of pressure ulcers, for example a viscoelastic gel. Preferably, the posterior plate 60 is multi-perforated in order to adapt the anchoring point of the tightening straps 57,58 to the dimensions of the patient's dorsal thorax. According to another embodiment, the posterior plate forms a posterior shell that follows part of the posterolateral chest wall. Advantageously the tightening means are fixed on both sides of the posterior shell, for example by rings. Of course, it is possible to use this thoracic restriction device without a posterior plate. In this case, a single tightening strap advantageously surrounds the anterolateral and dorsal parts of the patient's chest, as shown in FIGS. 3 and 4.

In the example embodiment shown in FIG. 7, the anteroposterior thoracic restriction device 70 comprises a rigid or semi-rigid anterior plate 67 made of preferentially biocompatible material. A fluid bag 68 for application to the sternum of the patient's rib cage is attached to the inner wall (71) of the anterior plate 67. This attachment can be reversible or irreversible. The anterior plate 67 can integrate a pressure sensor 69 in direct communication with the bag 68. This pressure sensor 69 allows the pressure inside the fluid bag 68 to be displayed directly.

The anterior plate comprises lateral holding means 63, 64, 65, 66, attached to said plate. Preferably, these lateral holding means include reinforcements to stiffen them. According to an embodiment, the lateral holding means 63, 64 comprise attachment means 61, 62 intended to attach straps or shoulder straps to hold the device in the cranio-caudal direction.

In the example shown in FIG. 7, the reinforcements of the lateral holding means 63, 64, intended to be closest to the patient's shoulders, are L-shaped to provide means of attachment to the shoulder straps.

The lateral holding means 63, 64, 65, 66 extend from the anterior plate 67 on either side of said plate so as to extend perpendicularly to the patient's chest. In the example shown in FIG. 7, the lateral holding means 63, 64, 65, 66 are raised from the anterior plate.

The holding means and/or reinforcements advantageously include openings, intended for the introduction of tightening means.

Generally, the device according to the invention, when used in conjunction with a positive-pressure artificial ventilation system (invasive or noninvasive), limits the compliance of the anterior thoracic wall and thus limit the risk of overdistension of the anterior areas and promote the redistribution of the air insufflated into the posterior and inferior areas of the lungs (FIG. 5B). Conversely, in the absence of such a device (FIG. 5A), the distribution is essentially anterior, with little or no ventilation in the posterior and inferior areas.

### Proof of Concept

The proof of concept was performed on a Thiel cadaver model (“Thiel cadaver”) under invasive mechanical ventilation. Thiel cadavers have benefited from a special embalming method, allowing them to retain the natural elasticity of the tissue. Of particular interest, the lungs of mechanically ventilated Thiel cadavers behave in a manner similar to the lungs of an ARDS patient, with condensed posterior areas and aerated anterior areas. The respiratory mechanics parameters are thus comparable to those of an ARDS patient.

During the proof-of-concept stage, a thoracic electrical impedance tomography (EIT) device was applied to cadavers with or without an anteroposterior thoracic restriction device according to the invention as described in FIG. 4. EIT allows regional measurement of impedance changes, which correspond to changes in aeration during invasive mechanical ventilation. EIT therefore allows direct visualization of ventilated areas and regional quantification of the gain or loss of aeration after a procedure.

FIG. 8 describes a screenshot of the EIT of a mechanically ventilated corpse to which the device according to the invention has been applied.

Box 1 represents dynamic EIT recordings of the cadaver during the experimental design. The EIT reports regional changes in impedance on a cross-section of the thorax. For each section, the back is at the bottom and the anterior part of the chest is at the top. Section (C) shows, in white, the envelope of all the lung regions ventilated during insufflation of the ventilator without the device according to the invention. Section (A) shows, in white, the envelope of all ventilated lung regions appears during insufflation of the ventilator with the device according to the invention in place (pressure applied to the anterior chest wall: about 80 cm H₂O). Section (B) represents the regional differences in aeration between the experimental step of section (C), i.e. without the device according to the invention, and the experimental step of section (A), i.e. with the device according to the invention. The dark gray areas correspond to decreases in aeration between step (C) and step (A). The areas in light gray correspond to aeration gains between step (C) and step (A). It can be observed that the application of the device according to the invention leads to a decrease in the aeration of the anterior areas (which nevertheless remain ventilated as shown by the envelope of the ventilated areas on section (A)) and an increase in the aeration of the posterior areas with a gain in the total volume of the ventilated lung, corresponding to a recruitment of the previously nonaerated posterior areas.

Box 2 represents the overall impedance changes (top curve) and the impedance changes per region of interest, from the most anterior region (ROI 1) to the most posterior region (ROI 4), as a function of time, during the application of the device according to the invention. The four regions of interest correspond to the four rectangles numbered 1 to 4 on the EIT sections in Box 1. These impedance changes taken in isolation are difficult to interpret and one must refer to Box 3 to understand their significance.

Box 3 describes the regional proportions of impedance changes relative to the overall impedance change. It is therefore the percentage of the total ventilation that reaches a region of interest. In each region of interest, the figure in large print reports the percentage of the total ventilation with the device according to the invention in place; the figure below, in small print, reports the percentage of the total ventilation without the device according to the invention.

Thus, in the absence of a device according to the invention, 85% of the total ventilation is distributed in the anterior half of the thorax (ROI 1 and 2) and only 15% in the posterior half. With the application of the device according to the invention, 62% of the total ventilation is distributed in the anterior half and 38% in the posterior half.

The results described above therefore show that the use of the device according to the invention allows a reduction in ventilation in the anterior areas limiting the risk of overdistension, to the benefit of a gain in aeration in the posterior areas.

1-12. (canceled)

13. An anteroposterior thoracic restriction device (10, 20, 40, 50, 70) comprising holding means for surrounding a patient's chest, a compressible fluid bag (11) intended to be held against the patient's sternum by said holding means, and reversible bilateral tightening means (16, 17; 25, 26; 43, 44), arranged on either side of the fluid bag and capable of reversibly tightening the holding means around the patient's chest.

14. The anteroposterior thoracic restriction device as claimed in claim 13, wherein the holding means comprise a rigid or semi-rigid anterior plate (21, 41) intended to be applied against the anterior part of the patient's chest, and optionally a rigid or semi-rigid posterior plate (22) intended to be applied against the posterior part of the patient's chest.

15. The anteroposterior thoracic restriction device as claimed in claim 13, wherein the holding means comprises a strap (14), preferentially a semi-rigid strap.

16. The anteroposterior thoracic restriction device as claimed in claim 13, wherein the tightening means are progressive tightening means.

17. The anteroposterior thoracic restriction device as claimed in claim 13, wherein the tightening means are anterolateral and/or wherein the tightening means are capable of applying a pressure of between 20 and 150 cm of water (cmH₂O)±20 in the fluid bag, when this is held between the strap and the patient's chest.

18. The anteroposterior thoracic restriction device as claimed in claim 17, wherein the tightening means are anterolateral and/or wherein the tightening means are capable of applying a pressure of 60 cm of water (cmH₂O)±20 in the fluid bag, when this is held between the strap and the patient's chest.

19. The anteroposterior thoracic restriction device as claimed in claim 13, said device further comprising a pressure sensor for measuring the pressure in the fluid bag and/or automatic release means.

20. The anteroposterior thoracic restriction device as claimed in claim 13, wherein the fluid bag contains a liquid.

21. The anteroposterior thoracic restriction device as claimed in claim 20, wherein the liquid is water.

22. The anteroposterior thoracic restriction device as claimed in claim 20, wherein the amount and/or volume of said fluid is constant in the compressible fluid bag.

23. The anteroposterior thoracic restriction device as claimed in claim 13, wherein the anterior strap or plate comprises a central housing (18, 69) for receiving the fluid bag.

24. The anteroposterior thoracic restriction device as claimed in claim 13, wherein the size of the bag is substantially equal to the dimensions of the patient's sternum.

25. An artificial ventilation system comprising a ventilator connected to a nasal and/or buccal and/or tracheal interface, for supplying air to the lungs of a patient, said system further comprising an anteroposterior thoracic restriction device as claimed in claim 13.

26. An artificial ventilation kit comprising an artificial ventilation system as claimed in claim 25, and means for depressing at least part of the patient's chest.
